(19) United States
(12) Patent Application Publication
(10) Pub. No.: US 2025/0308636 A1
(43) Pub. Date: Oct. 2, 2025

(54) INFERRING CNVS FROM THE DISTRIBUTION OF MOLECULES IN HYPER PARTITION

(71) Applicant: GUARDANT HEALTH, INC., Palo Alto, CA (US)

(72) Inventors: Denis TOLKUNOV, Dublin, CA (US); Catalin BARBACIORU, Fremont, CA (US)

(21) Appl. No.: 19/091,512

(22) Filed: Mar. 26, 2025

Related U.S. Application Data
(60) Provisional application No. 63/570,443, filed on Mar. 27, 2024.

(51) Int. Cl. G16B 30/10; G16B 40/20

(52) U.S. Cl. CPC G16B 30/10; G16B 40/20

(57) ABSTRACT

Methods and systems are described for improving detection of copy number by distribution of molecules. In the context of applying a genomic and epigenomic panel, on target molecules are exceedingly sparse, whereas large structural genomic alterations like CNV require observations of broader regions of the genome. Bins for analyses can be generated that do not overlap with genomic and epigenomic panels, based on distribution of off-target molecules. Reference samples from a pool of samples generates reference background against which test samples are normalized. A CNV determination takes into account, the tumor fraction of a sample and noise levels.

### CROSS REFERENCED TO RELATED APPLICATIONS

This application claims the benefit of U.S. Provisional Patent Application No. 63/570,443 filed Mar. 27, 2024, which is incorporated by reference herein in its entirety.

### BACKGROUND

Genetic variants, such as insertions, deletions, substitutions, rearrangements and copy number variants may be correlated with disease and response to therapeutic intervention. Identifying genetic variants accurately is therefore becoming increasingly important for diagnosing and treating disease. Copy number variations (CNVs) can contribute to a wide range of diseases and disorders, and knowing a person's CNV status can help to improve the diagnosis, treatment, and prevention of these conditions.

The majority of currently used methods for CNV detection relies on genomic data, including the bioinformatic approaches that use genomic information by inferring CNVs from hypo-methylated molecules. Methylation data has largely been overlooked in the context of CNV detection. The Inventors have designed a computational approach that allows us to obtain the CNV signal from the DNA methylation data (hyper partition) by analyzing the distribution of hyper-methylated molecules in the off-target regions of the genomic/epigenomic panels. Described herein, using a selected set of clinical samples sequenced with the Infinity platform, that the large-scale CNVs derived from off-target hypermethylated molecules align with those detected from genomic data. This method for CNV detection using methylation data offers a wide range of practical applications: Detecting large scale CNVs in the future methylation-only products. Cancer subtyping using CNV signature patterns. Identification of HRD status from methylation data, existing methods are predominantly reliant on CNV detection based on genomic data. Enhancing our existing CNV detection algorithm based on genomics by incorporating methylation data as an additional, complementary source of signal. Possible QC applications for methylation-based assays.

### SUMMARY OF THE INVENTION

The disclosure relates detection and analyses of a genetic state of a locus of interest in genetic material. The genetic material may include Deoxyribonucleic Acid (DNA) or Ribonucleic Acid (RNA) from a genome, chromosome, or other genetic material of a sample. The genetic state may include a variation from a wildtype sequence of the nucleic acid sequenced from the sample. Such variation may include, without limitation, a copy number variant (CNV) (which may include a series of deletions also referred to as copy number loss (CNL) relative to the wildtype state or insertions), a rearrangement, and/or other states. Based on the diagnostic, one or more treatment options may be determined. However, other types of genetic states of other loci of interest may be modeled.

A method of determining copy number variation (CNV) in a sample, including obtaining or having obtained a biological sample from the subject, wherein the biological sample comprises cell-free deoxyribonucleic acid (cfDNA) molecules; performing or having performed a diagnostic assay on the biological sample, wherein the diagnostic assay includes obtaining a set of sequence reads from a plurality of polynucleotides derived from the cfDNA molecules and analyzing the sequence reads to obtain a quantitative measure of CNV in a portion of a genome of interest.

The method wherein the diagnostic assay includes ligating molecular barcodes to a plurality of the cfDNA molecules in the biological sample to generate tagged parent polynucleotides;

amplifying a plurality of tagged parent polynucleotides to generate amplified progeny polynucleotides; sequencing a plurality of the amplified progeny polynucleotides to generate the set of sequence reads, wherein the set of sequence reads comprises sequence information corresponding to a polynucleotide derived from the plurality of cfDNA molecules and sequence information from the molecular barcodes that were ligated to the cfDNA molecules.

The method wherein the diagnostic assay includes purifying cell-free nucleic acids from a sample; physically fractionating the cell-free nucleic acids to generate one or more partitions, wherein the physical fractionating comprises fractionating nucleic acids based on one or more characteristics, wherein the one or more characteristics comprises methylation status; and sequencing at least a fraction of nucleic acids in the one or more partitions to generate a set of sequencing reads. The method includes attaching NGS-enabling adapters comprising differential molecular tags to each of the one or more partitions to generate molecular tagged partitions. The method includes differential molecular tags are different sets of molecular tags corresponding to a partition. The method includes physically fractionating comprises fractionating with methyl-binding domain protein (“MBD”)-beads to stratify into various degrees of methylation. The method includes at least one partition comprises hypermethylated DNA. The method includes physically fractionating comprises separating DNA molecules using immunoprecipitation. The method includes various degrees of methylation that comprise hypermethylation and hypomethylation. The method includes, amplifying the cell-free nucleic acids from the one or more partitions to generate amplified nucleic acids. The method includes re-combining one or more molecular tagged partitions. The method includes enriching the re-combined one or more molecular tagged partitions for a plurality of genomic regions. The method includes aplurality of genomic regions comprises differentially methylated regions. The method includes enriching by hybridization of amplified nucleic acids to RNA or DNA probes.

The method includes analyses to obtain a quantitative measure of CNV by generating bins from sequence reads that are not otherwise included in a genomic or epigenomic panel. The method includes off-target reads. For example, bins for analyses can be generated that do not overlap with genomic and cpigenomic panels, based on distribution of off-target molecules. The method includes only those reads in the hyperpartition. Reference samples from a pool of samples generates reference background against which test samples are normalized. A CNV determination takes into account, the tumor fraction of a sample and noise levels. This includes, for example, maximizing positive predictive agreement (PPA) in a sample, using sets of overlapping reads found in hyper and hypo partitions, and also negative predictive agreement (NPA). PPA can be calculated on the basis of segments CN #2, in hyper and hypo partitions; NPA can be calculated on the basis of copy-neutral segments in hyper and hypo partitions.

Described herein is a method, comprising: obtaining sequence data comprising sequence representations related to a plurality of polynucleotide molecules in a test sample; generating, a set of aligned sequence representations by performing an alignment process that determines one or more of the sequence representations that have at least a threshold amount of homology with respect to a portion of a reference human genome; generating a set of off-target sequence representations by identifying a first portion of the number of aligned sequence representations that do not correspond to target regions of the reference human genome; generating a set of on-target sequence representations by identifying a second portion of the number of aligned sequence representations that correspond to the target regions of the reference human genome; determining a plurality of first quantitative measures for the set of off-target sequence representations; determining a plurality of second quantitative measures based on adjustment of one or more of the plurality of the first quantitative measures, wherein adjustment of the first quantitative measures includes comparison to a plurality of reference quantitative measures; determining a measurement of copy number variants (CNVs) for one or more of the individual segments of the set of off-target sequence representations based on individual second quantitative measures that correspond to the one or more of the individual segments.

In other embodiments, the sequence data indicating sequence representations related to polynucleotide molecules includes genomic sequence information. In other embodiments, the sequence data indicating sequence representations related to polynucleotide molecules includes epigenomic sequence information. In other embodiments, the sequence data indicating sequence representations related to polynucleotide molecules includes genomic and epigenomic sequence information. In other embodiments, the sequence data indicating sequence representations related to polynucleotide molecules includes genomic and epigenomic sequence information and sequence representations related to polynucleotides are from one or more loci, each of which are mutually exclusive to the genomic and the epigenomic sequence information. For example, the loci found in genomic sequence would not be the same as those in epigenomic sequence, and vice-versa. By extension, individual segments and bins would also not be in genomic sequence, in epigenomic sequence, and vice-versa. In other embodiments, the plurality of reference quantitative measures are for the set of off-target sequence representations. In other embodiments, the plurality of reference quantitative measures are for the set of on-target sequence representations. In other embodiments, the reference quantitative measure are generated from a plurality of reference samples. In other embodiments, the reference samples are from healthy subjects. In other embodiments, the test sample is from a subject that is healthy, suspected of a having disease such as cancer, afflicted with cancer, or another disease. In other embodiments, generating the reference quantitative measure includes normalizing to a median of medians for one or more molecules counts obtained from each sample in a plurality of samples. In other embodiments, generating the reference quantitative measure includes generating an expected log-number based on median of the log of normalized one or more molecule counts from a plurality of samples. In other embodiments, comparing to the plurality of reference quantitative measures includes normalizing to a median of medians for one or more molecules counts obtained from the test sample. In other embodiments, comparing to the plurality of reference quantitative measures includes subtraction of log 2 values for one or more molecules counts obtained from the plurality of samples from media-centered log 2 values for one or more molecule counts obtained from the test sample. In other embodiments, the one or more loci are in a bin of 1-10 kb, 10-20 kb, 20-30 kb, 30-40 kb, 40-50 kb, 50-60 kb, 60-70 kb, 70-80 kb, 80-90 kb, 90-100 kb, 100-110 kb, 110-120 kb, 120-130 kb, 130-140 kb, 140-150 kb, 150 kb or more. In other embodiments, the one or more loci are in a bin of 50-60 kb, 60-70 kb, 70-80 kb, 80-90 kb, 90-100 kb, 100-110 kb, 110-120 kb, 120-130 kb, 130-140 kb, 140-150 kb, 150-250 kb or more. In other embodiments, the one or more loci are within a bin. In other embodiments, the one or more individual segments are in a bin. In other embodiments, determining an estimate of copy number variants (CNVs) for one or individual segments of the set of off-target sequence representations includes comparison to a threshold. In other embodiments, the threshold is based on maximum positive predictive accuracy (PPA) of the test sample and/or one or more additional samples. In other embodiments, the threshold is based on maximum positive predictive accuracy (PPA) of the test sample and one or more additional samples. In other embodiments, determining an estimate of copy number variants (CNVs) for one or individual segments of the set of off-target sequence representations includes application of circular binary segmentation (CBS) to identify genomic segments of equal copy number. In other embodiments, the determination of HRD status in a sample based on the estimate of CNVs for one or individual segments of the set of off-target sequence representations.

For example, determination of HRD status can include a method of obtaining, by a computing system having one or more hardware processors and memory, training sequence data including training sequence representations derived from a plurality of samples, individual training sequence representations including a nucleotide sequence corresponding to a fragment of a nucleic acid included in a sample of a plurality of samples and individual samples of the plurality of samples corresponding to a subject classified as having a homologous recombination repair deficiency; determining, by the computing system, a subset of the training sequence representations that correspond to nucleic acids having at least a threshold amount of methylated cytosines in one or more regions of the nucleotide sequence; analyzing, by the computing system, the subset of training sequence representations to determine quantitative measures derived from the subset of the training sequence representations, which can include CNV estimates. Here, individual quantitative measures correspond to a classification region of a plurality of classification regions of a reference genome, individual classification regions of the plurality of classification regions having the threshold amount of methylated cytosines in subjects in which cancer is detected; analyzing, by the computing system and using one or more computational techniques, the quantitative measures of the plurality of classification regions to determine a subset of the plurality of classification regions having at least a threshold likelihood of indicating a homology directed repair deficiency; and generating, by the computing system, a predictive model to determine a probability of a homologous recombination repair deficiency being present in one or more additional subjects, the predictive model including a plurality of variables and a plurality of weights with individual weights of the plurality of weights corresponding to individual variables of the plurality of variables, wherein an individual variable of the plurality of variables corresponds to an individual classification region of the subset of the plurality of classification regions and an individual weight that corresponds to the individual variable indicates a likelihood of the individual classification region indicating a homologous recombination repair deficiency.

Described herein is a system for performing a method, comprising: obtaining sequence data comprising sequence representations related to a plurality of polynucleotide molecules in a test sample; generating, a set of aligned sequence representations by performing an alignment process that determines one or more of the sequence representations that have at least a threshold amount of homology with respect to a portion of a reference human genome; generating a set of off-target sequence representations by identifying a first portion of the number of aligned sequence representations that do not correspond to target regions of the reference human genome; generating a set of on-target sequence representations by identifying a second portion of the number of aligned sequence representations that correspond to the target regions of the reference human genome; determining a plurality of first quantitative measures for the set of off-target sequence representations; determining a plurality of second quantitative measures based on adjustment of one or more of the plurality of the first quantitative measures, wherein adjustment of the first quantitative measures includes comparison to a plurality of reference quantitative measures; determining a measurement of copy number variants (CNVs) for one or more of the individual segments of the set of off-target sequence representations based on individual second quantitative measures that correspond to the one or more of the individual segments.

In other embodiments, the sequence data indicating sequence representations related to polynucleotide molecules includes genomic sequence information In other embodiments, the sequence data indicating sequence representations related to polynucleotide molecules includes epigenomic sequence information. In other embodiments, the sequence data indicating sequence representations related to polynucleotide molecules includes genomic and epigenomic sequence information. In other embodiments, the sequence data indicating sequence representations related to polynucleotide molecules includes genomic and epigenomic sequence information and sequence representations related to polynucleotides are from one or more loci, each of which are mutually exclusive to the genomic and the epigenomic sequence information. In other embodiments, the plurality of reference quantitative measures are for the set of off-target sequence representations. In other embodiments, the plurality of reference quantitative measures are for the set of on-target sequence representations. In other embodiments, the reference quantitative measure are generated from a plurality of reference samples. In other embodiments, the reference samples are from healthy subjects. In other embodiments, generating the reference quantitative measure includes normalizing to a median of medians for one or more molecules counts obtained from each sample in a plurality of samples. In other embodiments, generating the reference quantitative measure includes generating an expected log-number based on median of the log of normalized one or more molecule counts from a plurality of samples. In other embodiments, comparing to the plurality of reference quantitative measures includes normalizing to a median of medians for one or more molecules counts obtained from the test sample. In other embodiments, comparing to the plurality of reference quantitative measures includes subtraction of log 2 values for one or more molecules counts obtained from the plurality of samples from media-centered log 2 values for one or more molecule counts obtained from the test sample. In other embodiments, the one or more loci are in a bin of 1-10 kb, 10-20 kb, 20-30 kb, 30-40 kb, 40-50 kb, 50-60 kb, 60-70 kb, 70-80 kb, 80-90 kb, 90-100 kb, 100-110 kb, 110-120 kb, 120-130 kb, 130-140 kb, 140-150 kb, 150 kb or more. In other embodiments, determining an estimate of copy number variants (CNVs) for one or individual segments of the set of off-target sequence representations includes comparison to a threshold. In other embodiments, the threshold is based on maximum positive predictive accuracy (PPA) of the test sample and/or one or more additional samples. In other embodiments, the threshold is based on maxima positive predictive accuracy (PPA) of the test sample and one or more additional samples. In other embodiments, determining an estimate of copy number variants (CNVs) for one or individual segments of the set of off-target sequence representations includes application of circular binary segmentation (CBS) to identify genomic segments of equal copy number. In other embodiments, the determination of HRD status in a sample based on the estimate of CNVs for one or individual segments of the set of off-target sequence representations.

Described herein is a computer readable medium comprising instructions for performing a method, comprising: obtaining sequence data comprising sequence representations related to a plurality of polynucleotide molecules in a test sample; generating, a set of aligned sequence representations by performing an alignment process that determines one or more of the sequence representations that have at least a threshold amount of homology with respect to a portion of a reference human genome; generating a set of off-target sequence representations by identifying a first portion of the number of aligned sequence representations that do not correspond to target regions of the reference human genome; generating a set of on-target sequence representations by identifying a second portion of the number of aligned sequence representations that correspond to the target regions of the reference human genome; determining a plurality of first quantitative measures for the set of off-target sequence representations; determining a plurality of second quantitative measures based on adjustment of one or more of the plurality of the first quantitative measures, wherein adjustment of the first quantitative measures includes comparison to a plurality of reference quantitative measures; determining a measurement of copy number variants (CNVs) for one or more of the individual segments of the set of off-target sequence representations based on individual second quantitative measures that correspond to the one or more of the individual segments.

In other embodiments, the sequence data indicating sequence representations related to polynucleotide molecules includes genomic sequence information In other embodiments, the sequence data indicating sequence representations related to polynucleotide molecules includes epigenomic sequence information. In other embodiments, the sequence data indicating sequence representations related to polynucleotide molecules includes genomic and epigenomic sequence information. In other embodiments, the the sequence data indicating sequence representations related to polynucleotide molecules includes genomic and epigenomic sequence information and sequence representations related to polynucleotides are from one or more loci, each of which are mutually exclusive to the genomic and the epigenomic sequence information. In other embodiments, the the plurality of reference. In other embodiments, the plurality of reference quantitative measures are for the set of on-target sequence representations. In other embodiments, the reference quantitative measure are generated from a plurality of reference samples. In other embodiments, the reference samples are from healthy subjects. In other embodiments, generating the reference quantitative measure includes normalizing to a median of medians for one or more molecules counts obtained from each sample in a plurality of samples. In other embodiments, generating the reference quantitative measure includes generating an expected log-number based on median of the log of normalized one or more molecule counts from a plurality of samples. In other embodiments, comparing to the plurality of reference quantitative measures includes normalizing to a median of medians for one or more molecules counts obtained from the test sample. In other embodiments, comparing to the plurality of reference quantitative measures includes subtraction of log 2 values for one or more molecules counts obtained from the plurality of samples from media-centered log 2 values for one or more molecule counts obtained from the test sample. In other embodiments, the one or more loci are in a bin of 1-10 kb, 10-20 kb, 20-30 kb, 30-40 kb, 40-50 kb, 50-60 kb, 60-70 kb, 70-80 kb, 80-90 kb, 90-100 kb, 100-110 kb, 110-120 kb, 120-130 kb, 130-140 kb, 140-150 kb, 150 kb or more. In other embodiments, determining an estimate of copy number variants (CNVs) for one or individual segments of the set of off-target sequence representations includes comparison to a threshold. In other embodiments, the threshold is based on maximum positive predictive accuracy (PPA) of the test sample and/or one or more additional samples. In other embodiments, the threshold is based on maxima positive predictive accuracy (PPA) of the test sample and one or more additional samples. In other embodiments, determining an estimate of copy number variants (CNVs) for one or individual segments of the set of off-target sequence representations includes application of circular binary segmentation (CBS) to identify genomic segments of equal copy number. In other embodiments, the determination of HRD status in a sample based on the estimate of CNVs for one or individual segments of the set of off-target sequence representations.

### BRIEF DESCRIPTION OF THE FIGURES

FIG. 1.—Comparison of CNV segments detected in hyper partition vs. hypo partition BIP CNV caller segments

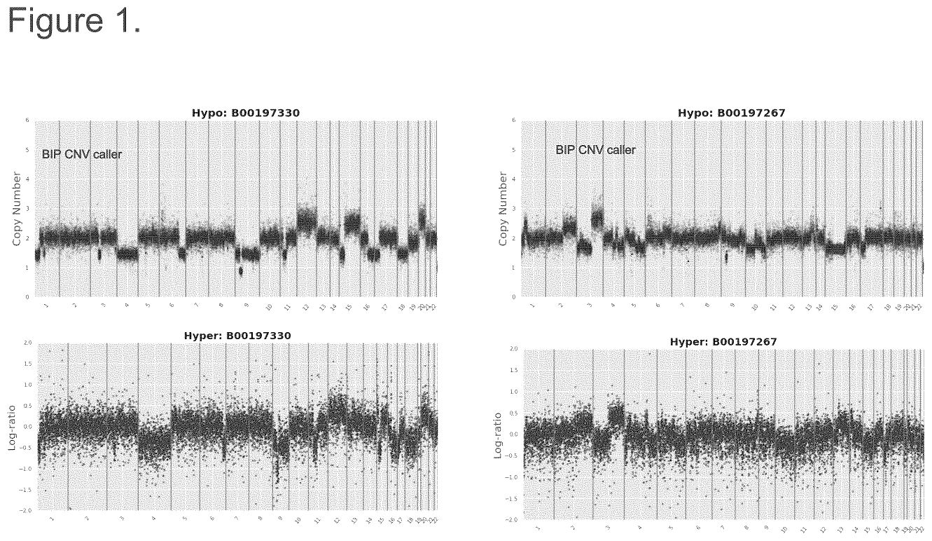

FIG. 2.—Comparison of CNV segments detected in hyper partition vs. hypo partition BIP CNV caller segments

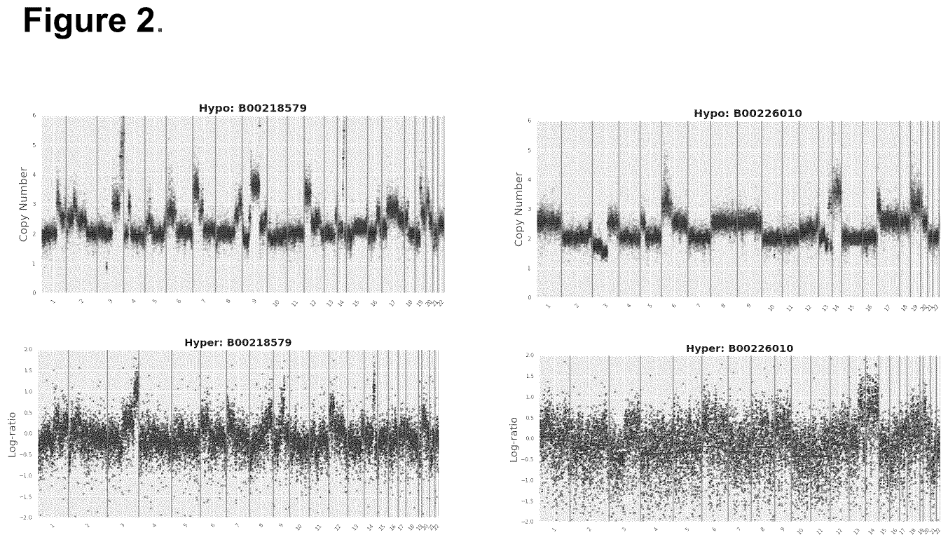

FIG. 3.—Comparison of CNV segments detected in hyper partition vs. hypo partition BIP CNV caller segments

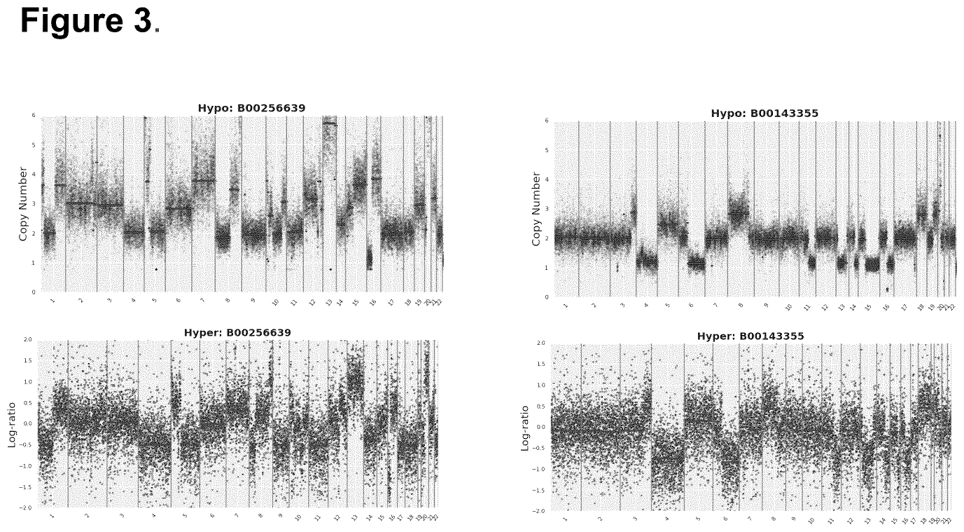

FIG. 4.—Assessing the performance. To derive CNV calls from log-ratios one utilizes a calling threshold. The CNV calling threshold depends on the tumor fraction of the sample and the level of noise. One empirically determines a threshold by maximizing the positive prediction accuracy (PPA) for each sample (equation 1), negative prediction accuracy (NPA) is also shown (equation 2).

FIG. 5.—Concordance between Methylation-Based and Genomic-Based CNV Calls (BIP CNV caller). Shown are sensitivity percent agreement (PPA) and specificity negative percent agreement (NPA).

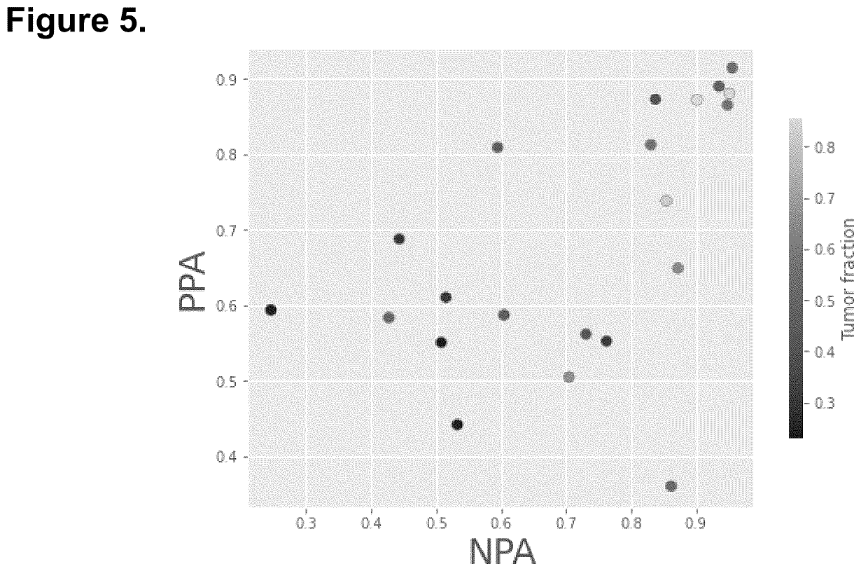

### DETAILED DESCRIPTION

### Computer Implementation

The present methods can be computer-implemented, such that any or all of the steps described in the specification or appended claims other than wet chemistry steps can be performed in a suitable programmed computer. The computer can be a mainframe, personal computer, tablet, smart phone, cloud, online data storage, remote data storage, or the like. The computer can be operated in one or more locations.

Various operations of the present methods can utilize information and/or programs and generate results that are stored on computer-readable media (e.g., hard drive, auxiliary memory, external memory, server; database, portable memory device (e.g., CD-R, DVD, ZIP disk, flash memory cards), and the like.

The present disclosure also includes an article of manufacture for analyzing a nucleic acid population that includes a machine-readable medium containing one or more programs which when executed implement the steps of the present methods.

The disclosure can be implemented in hardware and/or software. For example, different aspects of the disclosure can be implemented in either client-side logic or server-side logic. The disclosure or components thereof can be embodied in a fixed media program component containing logic instructions and/or data that when loaded into an appropriately configured computing device cause that device to perform according to the disclosure. A fixed media containing logic instructions can be delivered to a viewer on a fixed media for physically loading into a viewer's computer or a fixed media containing logic instructions may reside on a remote server that a viewer accesses through a communication medium to download a program component.

The code may be pre-compiled and configured for use with a machine having a processer adapted to execute the code, or can be compiled during runtime. The code can be supplied in a programming language that can be selected to enable the code to execute in a precompiled or as-compiled fashion.

Aspects of the systems and methods provided herein, such as the computer system 110, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk.

“Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible, storage media, “media” may include other types of (intangible) media.

“Storage” media, terms such as computer or machine “readable medium” refer to any tangible (such as physical), non-transitory, medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system can include or be in communication with an electronic display 935 that comprises a user interface (UI) for providing, for example, a report. Examples of UI's include, without limitation, a graphical user interface (GUI) and web-based user interface.

Methods and systems of the present disclosure can be implemented by way of one or more algorithms. An algorithm can be implemented by way of software upon execution by the processor 120.

### Sample Collection and Analysis Pipeline

A sample may be any biological sample isolated from a subject. Samples can include body tissues, such as known or suspected solid tumors, whole blood, platelets, serum, plasma, stool, red blood cells, white blood cells or leucocytes, endothelial cells, tissue biopsies, cerebrospinal fluid synovial fluid, lymphatic fluid, ascites fluid, interstitial or extracellular fluid, the fluid in spaces between cells, including gingival crevicular fluid, bone marrow, pleural effusions, cerebrospinal fluid, saliva, mucous, sputum, semen, sweat, urine. Samples are preferably body fluids, particularly blood and fractions thereof, and urine. Such samples include nucleic acids shed from tumors. The nucleic acids can include DNA and RNA and can be in double- and/or single-stranded forms. A sample can be in the form originally isolated from a subject or can have been subjected to further processing to remove or add components, such as cells, enrich for one component relative to another, or convert one form of nucleic acid to another, such as RNA to DNA or single-stranded nucleic acids to double-stranded. Thus, for example, a body fluid for analysis is plasma or serum containing cell-free nucleic acids, e.g., cell-free DNA (cfDNA).

In certain implementations, the polynucleotides can be enriched prior to sequencing. Enrichment can be performed for specific genomic target sequences. In certain embodiments, the specific genomic target sequences do not include the locus of interest. For example, the specific genomic target sequences may not include any portion of the locus of interest. In certain other implementations, enrichment can be performed nonspecifically. In some implementations, targeted regions of interest may be enriched with capture probes (“baits”) selected for one or more bait set panels using a differential tiling and capture scheme. A differential tiling and capture scheme uses bait sets of different relative concentrations to differentially tile (e.g., at different “resolutions”) across genomic regions associated with baits, subject to a set of constraints (e.g., sequencer constraints such as sequencing load, utility of each bait, etc.), and capture them at a desired level for downstream sequencing. These targeted genomic regions of interest may include regions of a subject's genome or transcriptome. In some implementations, biotin-labeled beads with probes to one or more regions of interest can be used to capture target sequences, optionally followed by amplification of those regions, to enrich for the regions of interest.

Sequence capture typically involves the use of oligonucleotide probes that hybridize to the target sequence. A probe set strategy can involve tiling the probes across a region of interest. Such probes can be, e.g., about 60 to 130 bases long. The set can have a depth of about 2×, 3×, 4×, 5×, 6×, 8×, 9×, 10×, 15×, 30×, 50×, or more. The effectiveness of sequence capture depends, in part, on the length of the sequence in the target molecule that is complementary (or nearly complementary) to the sequence of the probe.

In some implementations, the methods of the disclosure comprise selectively enriching regions from the subject's genome or transcriptome prior to sequencing. In other implementations, the methods of the disclosure comprise non-selectively enriching regions from the subject's genome or transcriptome prior to sequencing.

In certain implementations, sample index sequences are introduced to the polynucleotides after enrichment. The sample index sequences may be introduced through PCR or ligated to the polynucleotides, optionally as part of adapters.

The volume of plasma can depend on the desired read depth for sequenced regions. Exemplary volumes are 0.4-40 ml, 5-20 ml, 10-20 ml. For example, the volume can be 0.5 ml, 1 ml, 5 ml, 10 ml, 20 ml, 30 ml, or 40 ml. A volume of sampled plasma may be 5 to 20 ml.

The sample can comprise various amounts of nucleic acid that contains genome equivalents. For example, a sample of about 30 ng DNA can contain about 10,000 (10⁴) haploid human genome equivalents and, in the case of cfDNA, about 200 billion (2×10¹¹) individual polynucleotide molecules. Similarly, a sample of about 100 ng of DNA can contain about 30,000 haploid human genome equivalents and, in the case of cfDNA, about 600 billion individual molecules.

A sample can comprise nucleic acids from different sources, e.g., from cells and cell free. A sample can comprise nucleic acids carrying mutations. For example, a sample can comprise DNA carrying germline mutations and/or somatic mutations. A sample can comprise DNA carrying cancer-associated mutations (e.g., cancer-associated somatic mutations).

Exemplary amounts of cell free nucleic acids in a sample before amplification range from about 1 fg to about 1 μg, e.g., 1 μg to 200 ng, 1 ng to 100 ng, 10 ng to 1000 ng. For example, the amount can be up to about 600 ng, up to about 500 ng, up to about 400 ng, up to about 300 ng, up to about 200 ng, up to about 100 ng, up to about 50 ng, or up to about 20 ng of cell-free nucleic acid molecules. The amount can be at least 1 fg, at least 10 fg, at least 100 fg, at least 1 pg, at least 10 pg, at least 100 pg, at least 1 ng, at least 10 ng, at least 100 ng, at least 150 ng, or at least 200 ng of cell-free nucleic acid molecules. The amount can be up to 1 femtogram (fg), 10 fg, 100 fg, 1 picogram (pg), 10 μg, 100 pg, 1 ng, 10 ng, 100 ng, 150 ng, or 200 ng of cell-free nucleic acid molecules. The method can comprise obtaining 1 femtogram (fg) to 200 ng.

A cell-free nucleic acid sample refers to a sample containing cell-free nucleic acids. In some embodiments, “cell-free nucleic acids” refers to nucleic acids not contained within or otherwise bound to a cell at the point of isolation from the subject. Cell-free nucleic acids can be referred to all non-encapsulated nucleic acid sourced from a bodily fluid (e.g., blood, urine, CSF, etc.) from a subject. Cell-free nucleic acids include DNA (cfDNA), RNA (cfRNA), and hybrids thereof, including genomic DNA, mitochondrial DNA, circulating DNA, siRNA, miRNA, circulating RNA (cRNA), tRNA, rRNA, small nucleolar RNA (snoRNA), Piwi-interacting RNA (piRNA), long non-coding RNA (long ncRNA), or fragments of any of these. Cell-free nucleic acids can be double-stranded, single-stranded, or a hybrid thereof. A cell-free nucleic acid can be released into bodily fluid through secretion or cell death processes, e.g., cellular necrosis and apoptosis. Some cell-free nucleic acids are released into bodily fluid from cancer cells e.g., circulating tumor DNA (ctDNA). Others are released from healthy cells. ctDNA can be non-encapsulated tumor-derived fragmented DNA. Cell-free fetal DNA (cffDNA) is fetal DNA circulating freely in the maternal blood stream.

A cell-free nucleic acid or proteins associated with it can have one or more epigenetic modifications, for example, a cell-free nucleic acid can be acetylated, 5-methylated, ubiquitylated, phosphorylated, sumoylated, ribosylated, and/or citrullinated.

Cell-free nucleic acids have an exemplary size distribution of about 100-500 nucleotides, with molecules of 110 to about 230 nucleotides representing about 90% of molecules, with a mode of about 168 nucleotides in humans and a second minor peak in a range between 240 to 430 nucleotides. Cell-free nucleic acids can be about 160 to about 180 nucleotides, or about 320 to about 360 nucleotides, or about 430 to about 480 nucleotides.

Cell-free nucleic acids can be isolated from bodily fluids through a partitioning step in which cell-free nucleic acids, as found in solution, are separated from intact cells and other non-soluble components of the bodily fluid. Partitioning may include techniques such as centrifugation or filtration. Alternatively, cells in bodily fluids can be lysed and cell-free and cellular nucleic acids processed together. Generally, after addition of buffers and wash steps, cell-free nucleic acids can be precipitated with an alcohol. Further clean up steps may be used such as silica based columns to remove contaminants or salts. Non-specific bulk carrier nucleic acids, for example, may be added throughout the reaction to optimize certain aspects of the procedure such as yield.

After such processing, samples can include various forms of nucleic acid including double-stranded DNA, single stranded DNA and single stranded RNA. Optionally, single stranded DNA and RNA can be converted to double-stranded forms so they are included in subsequent processing and analysis steps.

### Amplification

Sample nucleic acids flanked by adapters can be amplified by PCR and other amplification methods typically primed from primers binding to primer binding sites in adapters flanking a DNA molecule to be amplified. Amplification methods can involve cycles of extension, denaturation and annealing resulting from thermocycling or can be isothermal as in transcription mediated amplification. Other amplification methods include the ligase chain reaction, strand displacement amplification, nucleic acid sequence based amplification, and self-sustained sequence based replication.

One or more amplifications can be applied to introduce barcodes to a nucleic acid molecule using conventional nucleic acid amplification methods. The amplification can be conducted in one or more reaction mixtures. Molecular barcodes and sample indexes can be introduced simultaneously, or in any sequential order. Molecular barcodes and sample indexes can be introduced prior to and/or after sequence capturing. In some cases, only the molecular barcodes are introduced prior to probe capturing while the sample indexes are introduced after sequence capturing. In some cases, both the molecular barcodes and the sample indexes are introduced prior to probe capturing. In some cases, the sample indexes are introduced after sequence capturing. Usually, sequence capturing involves introducing a single-stranded nucleic acid molecule complementary to a targeted sequence, e.g., a coding sequence of a genomic region and mutation of such region is associated with a cancer type. Typically, the amplifications generate a plurality of non-uniquely or uniquely tagged nucleic acid amplicons with molecular barcodes and sample indexes at a size ranging from 200 nt to 700 nt, 250 nt to 350 nt, or 320 nt to 550 nt. In some implementations, the amplicons have a size of about 300 nt. In some implementations, the amplicons have a size of about 500 nt.

### Barcodes

Barcodes can be incorporated into or otherwise joined to adapters by chemical synthesis, ligation, overlap extension PCR among other methods. Generally, assignment of unique or non-unique barcodes in reactions follows methods and systems described by U.S. patent applications Ser. No. 20/010,053519, 20110160078, and U.S. Pat. Nos. 6,582,908 and 7,537,898 and 9,598,731.

Tags can be linked to sample nucleic acids randomly or non-randomly. In some cases, they are introduced at an expected ratio of identifiers (e.g., a combination of barcodes) to microwells. The collection of barcodes can be unique, e.g., all the barcodes have different nucleotide sequence. The collection of barcodes can be non-unique, e.g., some of the barcodes have the same nucleotide sequence, and some of the barcodes have different nucleotide sequence. For example, the identifiers may be loaded so that more than 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 identifiers are loaded per genome sample. In some cases, the identifiers may be loaded so that less than 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 identifiers are loaded per genome sample. In some cases, the average number of identifiers loaded per sample genome is less than, or greater than, about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 identifiers per genome sample.

A preferred format uses 20-50 different tags, ligated to both ends of a target molecule creating 20-50×20-50 tags, e.g., 400-2500 tags combinations. Such numbers of tags are sufficient that different molecules having the same start and stop points have a high probability (e.g., at least 94%, 99.5%, 99.99%, 99.999%) of receiving different combinations of tags.

In some cases, identifiers may be predetermined or random or semi-random sequence oligonucleotides. In other cases, a plurality of barcodes may be used such that barcodes are not necessarily unique to one another in the plurality. In this example, barcodes may be attached (e.g., by ligation or PCR amplification) to individual molecules such that the combination of the barcode and the sequence it may be attached to creates a unique sequence that may be individually tracked. As described herein, detection of non-uniquely tagged barcodes in combination with sequence data of beginning (start) and end (stop) positions of sequence reads may allow assignment of a unique identity to a particular molecule. The length, or number of base pairs, of an individual sequence read may also be used to assign a unique identity to such a molecule. As described herein, fragments from a single strand of nucleic acid having been assigned a unique identity, may thereby permit subsequent identification of fragments from the parent strand, and/or a complementary strand.

### Sequencing Pipeline

Sample nucleic acids flanked by adapters with or without prior amplification can be subject to sequencing, such as by one or more sequencing devices 107. Sequencing methods include, for example, Sanger sequencing, high-throughput sequencing, pyrosequencing, sequencing-by-synthesis, single-molecule sequencing, nanopore sequencing, semiconductor sequencing, sequencing-by-ligation, sequencing-by-hybridization, RNA-Seq (Illumina), Digital Gene Expression (Helicos), Next generation sequencing, Single Molecule Sequencing by Synthesis (SMSS) (Helicos), massively-parallel sequencing, Clonal Single Molecule Array (Solexa), shotgun sequencing, Ion Torrent, Oxford Nanopore, Roche Genia, Maxim-Gilbert sequencing, primer walking, sequencing using PacBio, SOLID, Ion Torrent, or Nanopore platforms. Sequencing reactions can be performed in a variety of sample processing units, which may be multiple lanes, multiple channels, multiple wells, or other means of processing multiple sample sets substantially simultaneously. Sample processing unit can also include multiple sample chambers to enable processing of multiple runs simultaneously.

The sequencing reactions can be performed on one or more fragments types known to contain markers of cancer of other disease. The sequencing reactions can also be performed on any nucleic acid fragments present in the sample. The sequence reactions may provide for sequence coverage of the genome of at least 5%, 10%, 15%, 20%, 25%, 30%, 40%, 50%, 60%, 70%, 80%, 90%, 95%, 99%, 99.9% or 100%. In other cases, sequence coverage of the genome may be less than 5%, 10%, 15%, 20%, 25%, 30%, 40%, 50%, 60%, 70%, 80%, 90%, 95%, 99%, 99.9% or 100%.

Simultaneous sequencing reactions may be performed using multiplex sequencing. In some cases, cell free polynucleotides may be sequenced with at least 1000, 2000, 3000, 4000, 5000, 6000, 7000, 8000, 9000, 10000, 50000, 100,000 sequencing reactions. In other cases, cell free polynucleotides may be sequenced with less than 1000, 2000, 3000, 4000, 5000, 6000, 7000, 8000, 9000, 10000, 50000, 100,000 sequencing reactions. Sequencing reactions may be performed sequentially or simultaneously. Subsequent data analysis may be performed on all or part of the sequencing reactions. In some cases, data analysis may be performed on at least 1000, 2000, 3000, 4000, 5000, 6000, 7000, 8000, 9000, 10000, 50000, 100,000 sequencing reactions. In other cases, data analysis may be performed on less than 1000, 2000, 3000, 4000, 5000, 6000, 7000, 8000, 9000, 10000, 50000, 100,000 sequencing reactions. An exemplary read depth is 1000-50000 reads per locus (base).

### Sequence Analysis Pipeline

The present methods can be used to diagnose presence of conditions, particularly cancer, in a subject, to characterize conditions (e.g., staging cancer or determining heterogeneity of a cancer), monitor response to treatment of a condition, effect prognosis risk of developing a condition or subsequent course of a condition.

Various cancers may be detected using the present methods. Cancers cells, as most cells, can be characterized by a rate of turnover, in which old cells die and replaced by newer cells. Generally dead cells, in contact with vasculature in a given subject, may release DNA or fragments of DNA into the blood stream. This is also true of cancer cells during various stages of the disease. Cancer cells may also be characterized, dependent on the stage of the disease, by various genetic aberrations such as copy number variation as well as rare mutations. This phenomenon may be used to detect the presence or absence of cancers individuals using the methods and systems described herein.

The types and number of cancers that may be detected may include blood cancers, brain cancers, lung cancers, skin cancers, nose cancers, throat cancers, liver cancers, bone cancers, lymphomas, pancreatic cancers, skin cancers, bowel cancers, rectal cancers, thyroid cancers, bladder cancers, kidney cancers, mouth cancers, stomach cancers, solid state tumors, heterogeneous tumors, homogenous tumors, and the like.

Cancers can be detected from genetic variations including mutations, rare mutations, indels, copy number variations, transversions, translocations, inversion, deletions, aneuploidy, partial ancuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns.

Genetic data can also be used for characterizing a specific form of cancer. Cancers are often heterogeneous in both composition and staging. Genetic profile data may allow characterization of specific sub-types of cancer that may be important in the diagnosis or treatment of that specific sub-type. This information may also provide a subject or practitioner clues regarding the prognosis of a specific type of cancer and allow either a subject or practitioner to adapt treatment options in accord with the progress of the disease. Some cancers progress, becoming more aggressive and genetically unstable. Other cancers may remain benign, inactive, or dormant. The system and methods of this disclosure may be useful in determining disease progression.

The present analysis is also useful in determining the efficacy of a particular treatment option. Successful treatment options may increase the amount of copy number variation or rare mutations detected in a subject's blood if the treatment is successful as more cancers may die and shed DNA. In other examples, this may not occur. In another example, perhaps certain treatment options may be correlated with genetic profiles of cancers over time. This correlation may be useful in selecting a therapy. Additionally, if a cancer is observed to be in remission after treatment, the present methods can be used to monitor residual disease or recurrence of disease.

The present methods can also be used for detecting genetic variations in conditions other than cancer. Immune cells, such as B cells, may undergo rapid clonal expansion upon the presence certain diseases. Clonal expansions may be monitored using copy number variation detection and certain immune states may be monitored. In this example, copy number variation analysis may be performed over time to produce a profile of how a particular disease may be progressing. Copy number variation or even rare mutation detection may be used to determine how a population of pathogens are changing during the course of infection. This may be particularly important during chronic infections, such as HIV/AIDS or Hepatitis infections, whereby viruses may change life cycle state and/or mutate into more virulent forms during the course of infection. The present methods may be used to determine or profile rejection activities of the host body, as immune cells attempt to destroy transplanted tissue to monitor the status of transplanted tissue as well as altering the course of treatment or prevention of rejection.

### Determination of 5-Methylcytosine Pattern of Nucleic Acids

Bisulfite-based sequencing and variants thereof provides a means of determining the methylation pattern of a nucleic acid. In some embodiments, determining the methylation pattern includes distinguishing 5-methylcytosine (5mC) from non-methylated cytosine. In some embodiments, determining methylation pattern includes distinguishing N6-methyladenine from non-methylated adenine. In some embodiments, determining the methylation pattern includes distinguishing 5-hydroxymethylcytosine (5hmC), 5-formylcytosine (5fC), and 5-carboxylcytosine (5caC) from non-methylated cytosine. Examples of bisulfite sequencing include, but are not limited to oxidative bisulfite sequencing (OX-BS-seq), Tet-assisted bisulfite sequencing (TAB-seq), and reduced bisulfite sequencing (redBS-seq).

Oxidative bisulfite sequencing (OX-BS-seq) is used to distinguish between 5mC and 5hmC, by first converting the 5hmC to 5fC, and then proceeding with bisulfite sequencing as previously described. Tet-assisted bisulfite sequencing (TAB-seq) can also be used to distinguish 5mc and 5hmC. In TAB-seq, 5hmC is protected by glucosylation. A Tet enzyme is then used to convert 5mC to 5caC before proceeding with bisulfite sequencing, as previously described. Reduced bisulfite sequencing is used to distinguish 5fC from modified cytosines.

Generally, in bisulfite sequencing, a nucleic acid sample is divided into two aliquots and one aliquot is treated with bisulfite. The bisulfite converts native cytosine and certain modified cytosine nucleotides (e.g. 5-formylcytosine or 5-carboxylcytosine) to uracil whereas other modified cytosines (e.g., 5-methylcytosine, 5-hydroxylmethylcystosine) are not converted. Comparison of nucleic acid sequences of molecules from the two aliquots indicates which cytosines were and were not converted to uracils. Consequently, cytosines which were and were not modified can be determined. The initial splitting of the sample into two aliquots is disadvantageous for samples containing only small amounts of nucleic acids, and/or composed of heterogeneous cell/tissue origins such as bodily fluids containing cell-free DNA.

The present disclosure provides methods allowing bisulfite sequencing and variants thereof. These methods work by linking nucleic acids in a population to a capture moiety, i.e., a label that can be captured or immobilized. Capture moieties include, without limitation, biotin, avidin, streptavidin, a nucleic acid including a particular nucleotide sequence, a hapten recognized by an antibody, and magnetically attractable particles. The extraction moiety can be a member of a binding pair, such as biotin/streptavidin or hapten/antibody. In some embodiments, a capture moiety that is attached to an analyte is captured by its binding pair which is attached to an isolatable moiety, such as a magnetically attractable particle or a large particle that can be sedimented through centrifugation. The capture moiety can be any type of molecule that allows affinity separation of nucleic acids bearing the capture moiety from nucleic acids lacking the capture moiety. Exemplary capture moieties are biotin which allows affinity separation by binding to streptavidin linked or linkable to a solid phase or an oligonucleotide, which allows affinity separation through binding to a complementary oligonucleotide linked or linkable to a solid phase. Following linking of capture moieties to sample nucleic acids, the sample nucleic acids serve as templates for amplification. Following amplification, the original templates remain linked to the capture moieties, but amplicons are not linked to capture moieties.

The capture moiety can be linked to sample nucleic acids as a component of an adapter, which may also provide amplification and/or sequencing primer binding sites. In some methods, sample nucleic acids are linked to adapters at both ends, with both adapters bearing a capture moiety. Preferably any cytosine residues in the adapters are modified, such as by 5methylcytosine, to protect against the action of bisulfite. In some instances, the capture moieties are linked to the original templates by a cleavable linkage (e.g., photocleavable desthiobiotin-TEG or uracil residues cleavable with USER™ enzyme, Chem. Commun. (Camb). 2015 Feb. 21; 51 (15): 3266-3269), in which case the capture moieties can, if desired, be removed.

The amplicons are denatured and contacted with an affinity reagent for the capture tag. Original templates bind to the affinity reagent whereas nucleic acid molecules resulting from amplification do not. Thus, the original templates can be separated from nucleic acid molecules resulting from amplification.

Following separation or partition, the respective populations of nucleic acids (i.e., original templates and amplification products) can be subjected to bisulfite treatment with the original template population receiving bisulfite treatment and the amplification products not. Alternatively, the amplification products can be subjected to bisulfite treatment and the original template population is not. Following such treatment, the respective populations can be amplified (which in the case of the original template population converts uracils to thymines). The populations can also be subjected to biotin probe hybridization for enrichment. The respective populations are then analyzed and sequences compared to determine which cytosines were 5-methylated (or 5-hydroxylmethylated) in the original. Detection of a T nucleotide in the template population (corresponding to an unmethylated cytosine converted to uracil) and a C nucleotide at the corresponding position of the amplified population indicates an unmodified C. The presence of C's at corresponding positions of the original template and amplified populations indicates a modified C in the original sample.

In some embodiments, a method uses sequential DNA-seq and bisulfite-seq (BIS-seq) NGS library preparation of molecular tagged DNA libraries. This process is performed by labeling of adapters (e.g., biotin), DNA-seq amplification of whole library, parent molecule recovery (e.g. streptavidin bead pull down), bisulfite conversion and BIS-seq. In some embodiments, the method identifies 5-methylcytosine with single-base resolution, through sequential NGS-preparative amplification of parent library molecules with and without bisulfite treatment. This can be achieved by modifying the 5-methyl-ated NGS-adapters (directional adapters; Y-shaped/forked with 5-methylcytosine replacing) used in BIS-seq with a label (e.g., biotin) on one of the two adapter strands. Sample DNA molecules are adapter ligated, and amplified (e.g., by PCR). As only the parent molecules will have a labeled adapter end, they can be selectively recovered from their amplified progeny by label-specific capture methods (e.g., streptavidin-magnetic beads). As the parent molecules retain 5-methylation marks, bisulfite conversion on the captured library will yield single-base resolution 5-methylation status upon BIS-seq, retaining molecular information to corresponding DNA-seq. In some embodiments, the bisulfite treated library can be combined with a non-treated library prior to enrichment/NGS by addition of a sample tag DNA sequence in standard multiplexed NGS workflow. As with BIS-seq workflows, bioinformatics analysis can be carried out for genomic alignment and 5-methylated base identification. In sum, this method provides the ability to selectively recover the parent, ligated molecules, carrying 5-methylcytosine marks, after library amplification, thereby allowing for parallel processing for bisulfite converted DNA. This overcomes the destructive nature of bisulfite treatment on the quality/sensitivity of the DNA-seq information extracted from a workflow. With this method, the recovered ligated, parent DNA molecules (via labeled adapters) allow amplification of the complete DNA library and parallel application of treatments that elicit epigenetic DNA modifications. The present disclosure discusses the use of BIS-seq methods to identify cytosine5-methylation (5-methylcytosine), but this is not limiting. Variants of BIS-seq have been developed to identify hydroxymethylated cytosines (5hmC; OX-BS-seq, TAB-seq), formylcytosine (5fC; redBS-scq) and carboxylcytosines. These methodologies can be implemented with the sequential/parallel library preparation described herein.

### Alternative Methods of Modified Nucleic Acid Analysis

The disclosure provides alternative methods for analyzing modified nucleic acids (e.g., methylated, linked to histones and other modifications discussed above). In some such methods, a population of nucleic acids bearing the modification to different extents (e.g., 0, 1, 2, 3, 4, 5 or more methyl groups per nucleic acid molecule) is contacted with adapters before fractionation of the population depending on the extent of the modification. Adapters attach to either one end or both ends of nucleic acid molecules in the population. Preferably, the adapters include different tags of sufficient numbers that the number of combinations of tags results in a low probability e.g., 95, 99 or 99.9% of two nucleic acids with the same start and stop points receiving the same combination of tags. Following attachment of adapters, the nucleic acids are amplified from primers binding to the primer binding sites within the adapters. Adapters, whether bearing the same or different tags, can include the same or different primer binding sites, but preferably adapters include the same primer binding site. Following amplification, the nucleic acids are contacted with an agent that preferably binds to nucleic acids bearing the modification (such as the previously described such agents). The nucleic acids are separated into at least two partitions differing in the extent to which the nucleic acids bear the modification from binding to the agents. For example, if the agent has affinity for nucleic acids bearing the modification, nucleic acids overrepresented in the modification (compared with median representation in the population) preferentially bind to the agent, whereas nucleic acids underrepresented for the modification do not bind or are more easily eluted from the agent. Following separation, the different partitions can then be subject to further processing steps, which typically include further amplification, and sequence analysis, in parallel but separately. Sequence data from the different partitions can then be compared.

Nucleic acids can be linked at both ends to Y-shaped adapters including primer binding sites and tags. The molecules are amplified. The amplified molecules are then fractionated by contact with an antibody preferentially binding to 5-methylcytosine to produce two partitions. One partition includes original molecules lacking methylation and amplification copies having lost methylation. The other partition includes original DNA molecules with methylation. The two partitions are then processed and sequenced separately with further amplification of the methylated partition. The sequence data of the two partitions can then be compared. In this example, tags are not used to distinguish between methylated and unmethylated DNA but rather to distinguish between different molecules within these partitions so that one can determine whether reads with the same start and stop points are based on the same or different molecules.

The disclosure provides further methods for analyzing a population of nucleic acid in which at least some of the nucleic acids include one or more modified cytosine residues, such as 5-methylcytosine and any of the other modifications described previously. In these methods, the population of nucleic acids is contacted with adapters including one or more cytosine residues modified at the 5C position, such as 5-methylcytosine. Preferably all cytosine residues in such adapters are also modified, or all such cytosines in a primer binding region of the adapters are modified. Adapters attach to both ends of nucleic acid molecules in the population. Preferably, the adapters include different tags of sufficient numbers that the number of combinations of tags results in a low probability e.g., 95, 99 or 99.9% of two nucleic acids with the same start and stop points receiving the same combination of tags. The primer binding sites in such adapters can be the same or different, but are preferably the same. After attachment of adapters, the nucleic acids are amplified from primers binding to the primer binding sites of the adapters. The amplified nucleic acids are split into first and second aliquots. The first aliquot is assayed for sequence data with or without further processing. The sequence data on molecules in the first aliquot is thus determined irrespective of the initial methylation state of the nucleic acid molecules. The nucleic acid molecules in the second aliquot are treated with bisulfite. This treatment converts unmodified cytosines to uracils. The bisulfite treated nucleic acids are then subjected to amplification primed by primers to the original primer binding sites of the adapters linked to nucleic acid. Only the nucleic acid molecules originally linked to adapters (as distinct from amplification products thereof) are now amplifiable because these nucleic acids retain cytosines in the primer binding sites of the adapters, whereas amplification products have lost the methylation of these cytosine residues, which have undergone conversion to uracils in the bisulfite treatment. Thus, only original molecules in the populations, at least some of which are methylated, undergo amplification. After amplification, these nucleic acids are subject to sequence analysis. Comparison of sequences determined from the first and second aliquots can indicate among other things, which cytosines in the nucleic acid population were subject to methylation.

Partitioning the Sample into a Plurality of Subsamples; Aspects of Samples; Analysis of Epigenetic Characteristics

In certain embodiments described herein, a population of different forms of nucleic acids (e.g., hypermethylated and hypomethylated DNA in a sample, such as a captured set of cfDNA as described herein) can be physically partitioned based on one or more characteristics of the nucleic acids prior to further analysis, e.g., differentially modifying or isolating a nucleobase, tagging, and/or sequencing. This approach can be used to determine, for example, whether certain sequences are hypermethylated or hypomethylated. In some embodiments, hypermethylation variable epigenetic target regions are analyzed to determine whether they show hypermethylation characteristic of tumor cells and/or hypomethylation variable epigenetic target regions are analyzed to determine whether they show hypomethylation characteristic of tumor cells. Additionally, by partitioning a heterogeneous nucleic acid population, one may increase rare signals, e.g., by enriching rare nucleic acid molecules that are more prevalent in one fraction (or partition) of the population. For example, a genetic variation present in hyper-methylated DNA but less (or not) in hypomethylated DNA can be more easily detected by partitioning a sample into hyper-methylated and hypo-methylated nucleic acid molecules. By analyzing multiple fractions of a sample, a multi-dimensional analysis of a single locus of a genome or species of nucleic acid can be performed and hence, greater sensitivity can be achieved.

In some instances, a heterogeneous nucleic acid sample is partitioned into two or more partitions (e.g., at least 3, 4, 5, 6 or 7 partitions). In some embodiments, each partition is differentially tagged. Tagged partitions can then be pooled together for collective sample prep and/or sequencing. The partitioning-tagging-pooling steps can occur more than once, with each round of partitioning occurring based on a different characteristics (examples provided herein) and tagged using differential tags that are distinguished from other partitions and partitioning means.

Examples of characteristics that can be used for partitioning include sequence length, methylation level, nucleosome binding, sequence mismatch, immunoprecipitation, and/or proteins that bind to DNA. Resulting partitions can include one or more of the following nucleic acid forms: single-stranded DNA (ssDNA), double-stranded DNA (dsDNA), shorter DNA fragments and longer DNA fragments. In some embodiments, partitioning based on a cytosine modification (e.g., cytosine methylation) or methylation generally is performed and is optionally combined with at least one additional partitioning step, which may be based on any of the foregoing characteristics or forms of DNA. In some embodiments, a heterogeneous population of nucleic acids is partitioned into nucleic acids with one or more epigenetic modifications and without the one or more epigenetic modifications. Examples of epigenetic modifications include presence or absence of methylation; level of methylation; type of methylation (e.g., 5-methylcytosine versus other types of methylation, such as adenine methylation and/or cytosine hydroxymethylation); and association and level of association with one or more proteins, such as histones. Alternatively or additionally, a heterogeneous population of nucleic acids can be partitioned into nucleic acid molecules associated with nucleosomes and nucleic acid molecules devoid of nucleosomes. Alternatively or additionally, a heterogeneous population of nucleic acids may be partitioned into single-stranded DNA (ssDNA) and double-stranded DNA (dsDNA). Alternatively, or additionally, a heterogeneous population of nucleic acids may be partitioned based on nucleic acid length (e.g., molecules of up to 160 bp and molecules having a length of greater than 160 bp).

In some instances, each partition (representative of a different nucleic acid form) is differentially labelled, and the partitions are pooled together prior to sequencing. In other instances, the different forms are separately sequenced. In some embodiments, a population of different nucleic acids is partitioned into two or more different partitions. Each partition is representative of a different nucleic acid form, and a first partition (also referred to as a subsample) includes DNA with a cytosine modification in a greater proportion than a second subsample. Each partition is distinctly tagged. The first subsample is subjected to a procedure that affects a first nucleobase in the DNA differently from a second nucleobase in the DNA of the first subsample, wherein the first nucleobase is a modified or unmodified nucleobase, the second nucleobase is a modified or unmodified nucleobase different from the first nucleobase, and the first nucleobase and the second nucleobase have the same base pairing specificity. The tagged nucleic acids are pooled together prior to sequencing. Sequence reads are obtained and analyzed, including to distinguish the first nucleobase from the second nucleobase in the DNA of the first subsample, in silico. Tags are used to sort reads from different partitions. Analysis to detect genetic variants can be performed on a partition-by-partition level, as well as whole nucleic acid population level. For example, analysis can include in silico analysis to determine genetic variants, such as CNV, SNV, indel, fusion in nucleic acids in each partition. In some instances, in silico analysis can include determining chromatin structure. For example, coverage of sequence reads can be used to determine nucleosome positioning in chromatin. Higher coverage can correlate with higher nucleosome occupancy in genomic region while lower coverage can correlate with lower nucleosome occupancy or nucleosome depleted region (NDR).

Samples can include nucleic acids varying in modifications including post-replication modifications to nucleotides and binding, usually noncovalently, to one or more proteins.

In an embodiment, the population of nucleic acids is one obtained from a serum, plasma or blood sample from a subject suspected of having neoplasia, a tumor, or cancer or previously diagnosed with neoplasia, a tumor, or cancer. The population of nucleic acids includes nucleic acids having varying levels of methylation. Methylation can occur from any one or more post-replication or transcriptional modifications. Post-replication modifications include modifications of the nucleotide cytosine, particularly at the 5-position of the nucleobase, e.g., 5-methylcytosine, 5-hydroxymethylcytosine, 5-formylcytosine and 5-carboxylcytosine. The affinity agents can be antibodies with the desired specificity, natural binding partners or variants thereof (Bock et al., Nat Biotech 28:1106-1114 (2010); Song et al., Nat Biotech 29:68-72 (2011)), or artificial peptides selected e.g., by phage display to have specificity to a given target.

Examples of capture moieties contemplated herein include methyl binding domain (MBDs) and methyl binding proteins (MBPs) as described herein, including proteins such as MeCP2 and antibodies preferentially binding to 5-methylcytosine. Likewise, partitioning of different forms of nucleic acids can be performed using histone binding proteins which can separate nucleic acids bound to histones from free or unbound nucleic acids. Examples of histone binding proteins that can be used in the methods disclosed herein include RBBP4, RbAp48 and SANT domain peptides. Although for some affinity agents and modifications, binding to the agent may occur in an essentially all or none manner depending on whether a nucleic acid bears a modification, the separation may be one of degree. In such instances, nucleic acids overrepresented in a modification bind to the agent at a greater extent that nucleic acids underrepresented in the modification. Alternatively, nucleic acids having modifications may bind in an all or nothing manner. But then, various levels of modifications may be sequentially eluted from the binding agent.

For example, in some embodiments, partitioning can be binary or based on degree/level of modifications. For example, all methylated fragments can be partitioned from unmethylated fragments using methyl-binding domain proteins (e.g., MethylMiner Methylated DNA Enrichment Kit (ThermoFisher Scientific)). Subsequently, additional partitioning may involve cluting fragments having different levels of methylation by adjusting the salt concentration in a solution with the methyl-binding domain and bound fragments. As salt concentration increases, fragments having greater methylation levels are cluted. In some instances, the final partitions are representative of nucleic acids having different extents of modifications (overrepresentative or underrepresentative of modifications). Overrepresentation and underrepresentation can be defined by the number of modifications born by a nucleic acid relative to the median number of modifications per strand in a population. For example, if the median number of 5-methylcytosine residues in nucleic acid in a sample is 2, a nucleic acid including more than two 5-methylcytosine residues is overrepresented in this modification and a nucleic acid with 1 or zero 5-methylcytosine residues is underrepresented. The effect of the affinity separation is to enrich for nucleic acids overrepresented in a modification in a bound phase and for nucleic acids underrepresented in a modification in an unbound phase (i.e. in solution). The nucleic acids in the bound phase can be cluted before subsequent processing.

When using MethylMiner Methylated DNA Enrichment Kit (ThermoFisher Scientific) various levels of methylation can be partitioned using sequential clutions. For example, a hypomethylated partition (e.g., no methylation) can be separated from a methylated partition by contacting the nucleic acid population with the MBD from the kit, which is attached to magnetic beads. The beads are used to separate out the methylated nucleic acids from the non-methylated nucleic acids. Subsequently, one or more elution steps are performed sequentially to clute nucleic acids having different levels of methylation. For example, a first set of methylated nucleic acids can be cluted at a salt concentration of 160 mM or higher, e.g., at least 150 mM, at least 200 mM, at least 300 mM, at least 400 mM, at least 500 mM, at least 600 mM, at least 700 mM, at least 800 mM, at least 900 mM, at least 1000 mM, or at least 2000 mM. After such methylated nucleic acids are eluted, magnetic separation is once again used to separate higher levels of methylated nucleic acids from those with lower level of methylation. The elution and magnetic separation steps can repeat themselves to create various partitions such as a hypomethylated partition (representative of no methylation), a methylated partition (representative of low level of methylation), and a hyper methylated partition (representative of high level of methylation).

In some methods, nucleic acids bound to an agent used for affinity separation are subjected to a wash step. The wash step washes off nucleic acids weakly bound to the affinity agent. Such nucleic acids can be enriched in nucleic acids having the modification to an extent close to the mean or median (i.e., intermediate between nucleic acids remaining bound to the solid phase and nucleic acids not binding to the solid phase on initial contacting of the sample with the agent). The affinity separation results in at least two, and sometimes three or more partitions of nucleic acids with different extents of a modification. While the partitions are still separate, the nucleic acids of at least one partition, and usually two or three (or more) partitions are linked to nucleic acid tags, usually provided as components of adapters, with the nucleic acids in different partitions receiving different tags that distinguish members of one partition from another. The tags linked to nucleic acid molecules of the same partition can be the same or different from one another. But if different from one another, the tags may have part of their code in common so as to identify the molecules to which they are attached as being of a particular partition. For further details regarding portioning nucleic acid samples based on characteristics such as methylation, see WO2018/119452, which is incorporated herein by reference. In some embodiments, the nucleic acid molecules can be fractionated into different partitions based on the nucleic acid molecules that are bound to a specific protein or a fragment thereof and those that are not bound to that specific protein or fragment thereof.

Nucleic acid molecules can be fractionated based on DNA-protein binding. Protein-DNA complexes can be fractionated based on a specific property of a protein. Examples of such properties include various epitopes, modifications (e.g., histone methylation or acetylation) or enzymatic activity. Examples of proteins which may bind to DNA and serve as a basis for fractionation may include, but are not limited to, protein A and protein G. Any suitable method can be used to fractionate the nucleic acid molecules based on protein bound regions. Examples of methods used to fractionate nucleic acid molecules based on protein bound regions include, but are not limited to, SDS-PAGE, chromatin-immuno-precipitation (ChIP), heparin chromatography, and asymmetrical field flow fractionation (AF4).

In some embodiments, partitioning of the nucleic acids is performed by contacting the nucleic acids with a methylation binding domain (“MBD”) of a methylation binding protein (“MBP”). MBD binds to 5-methylcytosine (5mC). MBD is coupled to paramagnetic beads, such as Dynabeads® M-280 Streptavidin via a biotin linker. Partitioning into fractions with different extents of methylation can be performed by eluting fractions by increasing the NaCl concentration.

An exemplary method for molecular tag identification of MBD-bead partitioned libraries through NGS is as follows:

Physical partitioning of an extracted DNA sample (e.g., extracted blood plasma DNA from a human sample) using a methyl-binding domain protein-bead purification kit, saving all elutions from process for downstream processing.

Parallel application of differential molecular tags and NGS-enabling adapter sequences to each partition. For example, the hypermethylated, residual methylation (‘wash’), and hypomethylated partitions are ligated with NGS-adapters with molecular tags.

Re-combining all molecular tagged partitions, and subsequent amplification using adapter-specific DNA primer sequences.

Enrichment/hybridization of re-combined and amplified total library, targeting genomic regions of interest (e.g., cancer-specific genetic variants and differentially methylated regions).

Re-amplification of the enriched total DNA library, appending a sample tag. Different samples are pooled and assayed in multiplex on an NGS instrument.

Bioinformatics analysis of NGS data, with the molecular tags being used to identify unique molecules, as well deconvolution of the sample into molecules that were differentially MBD-partitioned. This analysis can yield information on relative 5-methylcytosine for genomic regions, concurrent with standard genetic sequencing/variant detection.

Examples of MBPs contemplated herein include, but are not limited to:
(a) MeCP2 is a protein preferentially binding to 5-methyl-cytosine over unmodified cytosine.
(b) RPL26, PRP8 and the DNA mismatch repair protein MHS6 preferentially bind to 5-hydroxymethyl-cytosine over unmodified cytosine.
(c) FOXK1, FOXK2, FOXP1, FOXP4 and FOXI3 preferably bind to 5-formyl-cytosine over unmodified cytosine (Iurlaro et al., Genome Biol. 14: R119 (2013)).
(d) Antibodies specific to one or more methylated nucleotide bases.

In general, elution is a function of number of methylated sites per molecule, with molecules having more methylation eluting under increased salt concentrations. To elute the DNA into distinct populations based on the extent of methylation, one can use a series of elution buffers of increasing NaCl concentration. Salt concentration can range from about 100 nM to about 2500 mM NaCl. In one embodiment, the process results in three (3) partitions. Molecules are contacted with a solution at a first salt concentration and including a molecule including a methyl binding domain, which molecule can be attached to a capture moiety, such as streptavidin. At the first salt concentration a population of molecules will bind to the MBD and a population will remain unbound. The unbound population can be separated as a “hypomethylated” population. For example, a first partition representative of the hypomethylated form of DNA is that which remains unbound at a low salt concentration, e.g., 100 mM or 160 mM. A second partition representative of intermediate methylated DNA is eluted using an intermediate salt concentration, e.g., between 100 mM and 2000 mM concentration. This is also separated from the sample. A third partition representative of hypermethylated form of DNA is eluted using a high salt concentration, e.g., at least about 2000 mM.

The disclosure provides further methods for analyzing a population of nucleic acids in which at least some of the nucleic acids include one or more modified cytosine residues, such as 5-methylcytosine and any of the other modifications described previously. In these methods, after partitioning, the subsamples of nucleic acids are contacted with adapters including one or more cytosine residues modified at the 5C position, such as 5-methylcytosine. Preferably all cytosine residues in such adapters are also modified, or all such cytosines in a primer binding region of the adapters are modified. Adapters attach to both ends of nucleic acid molecules in the population. Preferably, the adapters include different tags of sufficient numbers that the number of combinations of tags results in a low probability e.g., 95, 99 or 99.9% of two nucleic acids with the same start and stop points receiving the same combination of tags. The primer binding sites in such adapters can be the same or different, but are preferably the same. After attachment of adapters, the nucleic acids are amplified from primers binding to the primer binding sites of the adapters. The amplified nucleic acids are split into first and second aliquots. The first aliquot is assayed for sequence data with or without further processing. The sequence data on molecules in the first aliquot is thus determined irrespective of the initial methylation state of the nucleic acid molecules. The nucleic acid molecules in the second aliquot are subjected to a procedure that affects a first nucleobase in the DNA differently from a second nucleobase in the DNA, wherein the first nucleobase includes a cytosine modified at the 5 position, and the second nucleobase includes unmodified cytosine. This procedure may be bisulfite treatment or another procedure that converts unmodified cytosines to uracils. The nucleic acids subjected to the procedure are then amplified with primers to the original primer binding sites of the adapters linked to nucleic acid. Only the nucleic acid molecules originally linked to adapters (as distinct from amplification products thereof) are now amplifiable because these nucleic acids retain cytosines in the primer binding sites of the adapters, whereas amplification products have lost the methylation of these cytosine residues, which have undergone conversion to uracils in the bisulfite treatment. Thus, only original molecules in the populations, at least some of which are methylated, undergo amplification. After amplification, these nucleic acids are subject to sequence analysis. Comparison of sequences determined from the first and second aliquots can indicate among other things, which cytosines in the nucleic acid population were subject to methylation.

Such an analysis can be performed using the following exemplary procedure. After partitioning, methylated DNA is linked to Y-shaped adapters at both ends including primer binding sites and tags. The cytosines in the adapters are modified at the 5 position (e.g., 5-methylated). The modification of the adapters serves to protect the primer binding sites in a subsequent conversion step (e.g., bisulfite treatment, TAP conversion, or any other conversion that does not affect the modified cytosine but affects unmodified cytosine). After attachment of adapters, the DNA molecules are amplified. The amplification product is split into two aliquots for sequencing with and without conversion. The aliquot not subjected to conversion can be subjected to sequence analysis with or without further processing. The other aliquot is subjected to a procedure that affects a first nucleobase in the DNA differently from a second nucleobase in the DNA, wherein the first nucleobase includes a cytosine modified at the 5 position, and the second nucleobase includes unmodified cytosine. This procedure may be bisulfite treatment or another procedure that converts unmodified cytosines to uracils. Only primer binding sites protected by modification of cytosines can support amplification when contacted with primers specific for original primer binding sites. Thus, only original molecules and not copies from the first amplification are subjected to further amplification. The further amplified molecules are then subjected to sequence analysis. Sequences can then be compared from the two aliquots. As in the separation scheme discussed above, nucleic acid tags in adapters are not used to distinguish between methylated and unmethylated DNA but to distinguish nucleic acid molecules within the same partition.

Subjecting the First Subsample to a Procedure that Affects a First Nucleobase in the DNA Differently from a Second Nucleobase in the DNA of the First Subsample

Methods disclosed herein comprise a step of subjecting the first subsample to a procedure that affects a first nucleobase in the DNA differently from a second nucleobase in the DNA of the first subsample, wherein the first nucleobase is a modified or unmodified nucleobase, the second nucleobase is a modified or unmodified nucleobase different from the first nucleobase, and the first nucleobase and the second nucleobase have the same base pairing specificity. In some embodiments, if the first nucleobase is a modified or unmodified adenine, then the second nucleobase is a modified or unmodified adenine; if the first nucleobase is a modified or unmodified cytosine, then the second nucleobase is a modified or unmodified cytosine; if the first nucleobase is a modified or unmodified guanine, then the second nucleobase is a modified or unmodified guanine; and if the first nucleobase is a modified or unmodified thymine, then the second nucleobase is a modified or unmodified thymine (where modified and unmodified uracil are encompassed within modified thymine for the purpose of this step).

In some embodiments, the first nucleobase is a modified or unmodified cytosine, then the second nucleobase is a modified or unmodified cytosine. For example, first nucleobase may comprise unmodified cytosine (C) and the second nucleobase may comprise one or more of 5-methylcytosine (mC) and 5-hydroxymethylcytosine (hmC). Alternatively, the second nucleobase may comprise C and the first nucleobase may comprise one or more of mC and hmC. Other combinations are also possible, as indicated, e.g., in the Summary above and the following discussion, such as where one of the first and second nucleobases includes mC and the other includes hmC.

In some embodiments, the procedure that affects a first nucleobase in the DNA differently from a second nucleobase in the DNA of the first subsample includes bisulfite conversion. Treatment with bisulfite converts unmodified cytosine and certain modified cytosine nucleotides (e.g. 5-formyl cytosine (fC) or 5-carboxylcytosine (caC)) to uracil whereas other modified cytosines (e.g., 5-methylcytosine, 5-hydroxylmethylcystosine) are not converted. Thus, where bisulfite conversion is used, the first nucleobase includes one or more of unmodified cytosine, 5-formyl cytosine, 5-carboxylcytosine, or other cytosine forms affected by bisulfite, and the second nucleobase may comprise one or more of mC and hmC, such as mC and optionally hmC. Sequencing of bisulfite-treated DNA identifies positions that are read as cytosine as being mC or hmC positions. Meanwhile, positions that are read as T are identified as being T or a bisulfite-susceptible form of C, such as unmodified cytosine, 5-formyl cytosine, or 5-carboxylcytosine. Performing bisulfite conversion on a first subsample as described herein thus facilitates identifying positions containing mC or hmC using the sequence reads obtained from the first subsample. For an exemplary description of bisulfite conversion, see, e.g., Moss et al., Nat Commun. 2018; 9:5068 . . .

In some embodiments, the procedure that affects a first nucleobase in the DNA differently from a second nucleobase in the DNA of the first subsample includes oxidative bisulfite (Ox-BS) conversion. In some embodiments, the procedure that affects a first nucleobase in the DNA differently from a second nucleobase in the DNA of the first subsample includes Tet-assisted bisulfite (TAB) conversion. In some embodiments, the procedure that affects a first nucleobase in the DNA differently from a second nucleobase in the DNA of the first subsample includes Tet-assisted conversion with a substituted borane reducing agent, optionally wherein the substituted borane reducing agent is 2-picoline borane, borane pyridine, tert-butylamine borane, or ammonia borane. In some embodiments, the procedure that affects a first nucleobase in the DNA differently from a second nucleobase in the DNA of the first subsample includes chemical-assisted conversion with a substituted borane reducing agent, optionally wherein the substituted borane reducing agent is 2-picoline borane, borane pyridine, tert-butylamine borane, or ammonia borane. In some embodiments, the procedure that affects a first nucleobase in the DNA differently from a second nucleobase in the DNA of the first subsample includes APOBEC-coupled epigenetic (ACE) conversion.

In some embodiments, procedure that affects a first nucleobase in the DNA differently from a second nucleobase in the DNA of the first subsample includes enzymatic conversion of the first nucleobase, e.g., as in EM-Seq. See, e.g., Vaisvila R, et al. (2019) EM-seq: Detection of DNA methylation at single base resolution from picograms of DNA. bioRxiv; DOI: 10.1101/2019.12.20.884692, available at www.biorxiv.org/content/10.1101/2019.12.20.884692v1. For example, TET2 and T4-βGT can be used to convert 5mC and 5hmC into substrates that cannot be deaminated by a deaminase (e.g., APOBEC3A), and then a deaminase (e.g., APOBEC3A) can be used to deaminate unmodified cytosines converting them to uracils.

In some embodiments, the procedure that affects a first nucleobase in the DNA differently from a second nucleobase in the DNA of the first subsample includes separating DNA originally including the first nucleobase from DNA not originally including the first nucleobase.

In some embodiments, the first nucleobase is a modified or unmodified adenine, and the second nucleobase is a modified or unmodified adenine. In some embodiments, the modified adenine is N6-methyladenine (mA). In some embodiments, the modified adenine is one or more of N6-methyladenine (mA), N6-hydroxymethyladenine (hmA), or N6-formyladenine (fA).

Techniques including methylated DNA immunoprecipitation (MeDIP) can be used to separate DNA containing modified bases such as mA from other DNA. See, e.g., Kumar et al., Frontiers Genet. 2018; 9:640; Greer et al., Cell 2015; 161:868-878. An antibody specific for mA is described in Sun et al., Bioessays 2015; 37:1155-62. Antibodies for various modified nucleobases, such as forms of thymine/uracil including halogenated forms such as 5-bromouracil, are commercially available. Various modified bases can also be detected based on alterations in their base-pairing specificity. For example, hypoxanthine is a modified form of adenine that can result from deamination and is read in sequencing as a G. See, e.g., U.S. Pat. No. 8,486,630; Brown, Genomes, 2nd Ed., John Wiley & Sons, Inc., New York, N.Y., 2002, chapter 14, “Mutation, Repair, and Recombination.”

### EXAMPLES

### Example 1—Problem

Copy number variations (CNVs) can contribute to a wide range of diseases and disorders, and knowing a person's CNV status can help to improve the diagnosis, treatment, and prevention of these conditions.

The majority of currently used methods for CNV detection relies on genomic data, including a bioinformatic CNV caller that is using genomic information by inferring CNVs from hypo-methylated molecules. More information is found in PCT App. No. PCT/US2022/071059, PCT/US2024/050063, PCT/US2016/067356, PCT/US2020/016120, PCT/US2021/048035, PCT/US2024/034033, PCT/US2023/084916, U.S. Prov. App. No. 63/724,549, each of which are fully incorporated by reference herein. In contrast, methylation data has largely been overlooked in the context of CNV detection. The Inventors have designed a computational approach that allows us to obtain the CNV signal from the DNA methylation data (hyper partition) by analyzing the distribution of hyper-methylated molecules in the off-target regions of the genomic/epigenomic panels.

Described herein, using a selected set of clinical samples sequenced with the Infinity platform, that the large-scale CNVs derived from off-target hypermethylated molecules align with those detected from genomic data. This method for CNV detection using methylation data offers a wide range of practical applications.

### Example 2—Main Idea

The distribution of molecules in hyper-partition is affected by methylation status of the DNA regions, by the copy number variations as well as by panel capture efficiency, GC-content and other biases.

Without being bound by any particular hypothesis, the Inventors utilized the effect that DNA methylation is relatively local event that affects the genes or genomic regions in proximity to the methylated CpG sites. Large structural genomic alterations like CNVs affect broader regions of the genome. Therefore, one can compute the coverage average over extensive genomic regions, the influence of CNVs will be more pronounced in comparison to methylation.

### Example 3—Method

Normalization of on-target coverage in hyper-partition is challenging primarily because the distribution in normal samples is exceedingly sparse, which can be a consequence of the epigenomic panel's design. Therefore, one utilizes the distribution of the off-target molecules. The Inventors constructed off-target bins of size 100 kb that do not overlap with genomic and epigenomic panels, excluding bad bins (based on the distribution of molecules in hypo partition) are discarded. As one example, a number of off-target bins after the filtering 17,898. To account for the variability of the coverage across the bins we build a reference using a pool of normal control samples.

### Example 4—Construction of a CNV Reference Using Off-Target Molecules in Hyper Partition

The Inventors selected 38 normal cfDNA samples to generate a reference background The Inventors ensured that the MBD binding curves and fragment size distributions exhibit a high degree of similarity across the samples. Each sample is normalized to the median of medians of hyper molecule counts across all autosomes. For each bin the expected log-number of molecules is calculated as the median of the logarithms of the normalized counts across the pool of 38 normal samples. The reliability of the bins is evaluated using a standard deviation of the normalized counts across the pool of 38 normal samples.

### Example 5—Normalization of Test Samples to the Reference

For the purpose of testing of our method, the Inventors selected 21 cfDNA samples with tumor fraction above 20%, with MBD binding curves exhibiting a high degree of similarity to the normal control samples.

Each sample is normalized to the median of medians of hyper molecule counts across all autosomes. Normalization to the CNV reference is achieved by subtracting the log 2 values of the reference from the median-centered log 2 counts for each bin. The Inventors use circular binary segmentation (CBS) implemented by the DNAcopy package to identify genomic segments of equal copy number.

### Example 6—Performance

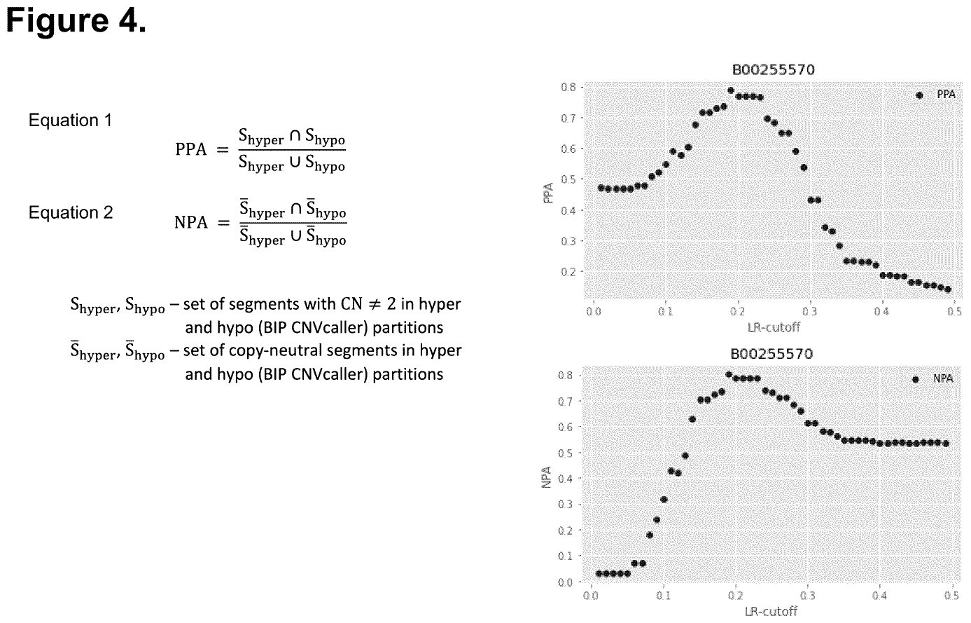

To derive CNV calls from log-ratios one can define a calling threshold empirically. The CNV calling threshold depends on the tumor fraction of the sample and the level of noise. Here, one can empirically find the thresholds by maximizing the PPA for each sample as shown in FIG. 4.

### Example 7—Detecting Large Scale CNVs in the Future Methylation-Only Products

One can utilize the aforementioned method and compositions to detect large scale CNVs, including in methylation (e.g., epigenomic) applications. For example, given the use of binning from off-target sequences, which are plentiful, the use of panel-based methylation detection leaves ample room to perform large scale CNV detection across a genome of interest. As such, this approach lends itself readily to being a complementary detection scheme when performing the aforementioned epigenomic analyses.

### Example 8—Cancer Subtyping Using CNV Signature Patterns

The aforementioned methods and compositions can also be used for detecting CNV signature and associations with cancer subtypes. Given the complementary nature of detection as described above, large scale CNV detection allow generation of CNV signatures. Given the known capability of methylation to classify and identify cancer subtypes, one can therefore associate a detected CNV signature and that which is concordant with a methylation pattern of a cancer subtype. In some instances, this may be detected in a single sample. In others, the concordance may be determined through a series of determinations across a plurality of samples.

### Example 9—Identification of HRD Status from Methylation Data

A particular application of the aforementioned methods and compositions is for determining HRD status in a sample obtained from a subject. Conventional existing methods are predominantly reliant on CNV detection based on genomic data. Here, one can utilize the complementary nature of CNV detection, in combination with the advantages brought by methylation (e.g., epigenomic) detection for identifying HRD status. The large scale nature of CNV detection use the aforementioned method provides a wider swath of measurements beyond genomic measurement alone. This increased range of CNV detection provides basis for a more informative approach of HRD status.

### Example 10—Enhancing Our CNV Detection Algorithm Based on Genomics by Incorporating methylation Data as an Additional, Complementary Source of Signal

As described, the utilization of the aforementioned methods and compositions readily lends itself to a complimentary detection scheme to methylation (e.g., epigenomic) detection. Given the simultaneous detection of both features, they can further improve detection of CNV using genomic methods by providing an orthogonal measurement to assess performance of CNV detection or perform as an enhancement of genomic based CNV detection.

### Example 11—Possible QC Applications for Methylation-Based Assays

Given the aforementioned methods and compositions, additional sample measurements will lead to repositories of CNV measurements with a primary example being the reference measurements obtained from a plurality of subjects. Eventually, such repositories of both reference and test samples provided basis for assessing test samples, as a form of quality control. An illustrative example includes a CNV measurement far beyond measurements observed in said repositories, leading one to potentially identify the presence of contaminants leading to increased copy number.

### Example 12—Future Improvements

Establish rigorous QC metrics for sample screening, ensuring the method's reliability in generating results. Implement bias correction e.g. GC-bias, fragment size distribution, MBD-based normalization. Use robust metric for the reference generation. Implement denoising steps, such as a more rigorous off-target bins selection. Discard “noisy” molecules with less than 10 CpGs.

1. A method, comprising:
obtaining sequence data comprising sequence representations related to a plurality of polynucleotide molecules in a test sample;
generating, a set of aligned sequence representations by performing an alignment process that determines one or more of the sequence representations that have at least a threshold amount of homology with respect to a portion of a reference human genome;
generating a set of off-target sequence representations by identifying a first portion of the number of aligned sequence representations that do not correspond to target regions of the reference human genome;
generating a set of on-target sequence representations by identifying a second portion of the number of aligned sequence representations that correspond to the target regions of the reference human genome;
determining a plurality of first quantitative measures for the set of off-target sequence representations;
determining a plurality of second quantitative measures based on adjustment of one or more of the plurality of the first quantitative measures, wherein adjustment of the first quantitative measures comprises comparison to a plurality of reference quantitative measures;
determining a measurement of copy number variants (CNVs) for one or more of the individual segments of the set of off-target sequence representations based on individual second quantitative measures that correspond to the one or more of the individual segments.

2. The method of claim 1, wherein the sequence data indicating sequence representations related to polynucleotide molecules comprises genomic sequence information.

3. The method of claim 1, wherein the sequence data indicating sequence representations related to polynucleotide molecules comprises epigenomic sequence information.

4. The method of claim 1, wherein the sequence data indicating sequence representations related to polynucleotide molecules comprises genomic and epigenomic sequence information.

5. The method of claim 1, wherein the sequence data indicating sequence representations related to polynucleotide molecules comprises genomic and epigenomic sequence information and sequence representations related to polynucleotides are from one or more loci, each of which are mutually exclusive to the genomic and the epigenomic sequence information.

6. The method of claim 1, wherein the plurality of reference quantitative measures are for the set of off-target sequence representations.

7. The method of claim 1, wherein the plurality of reference quantitative measures are for the set of on-target sequence representations.

8. The method of claim 1, wherein the reference quantitative measure are generated from a plurality of reference samples.

9. The method of claim 7, wherein the reference samples are from healthy subjects.

10. The method of claim 1, wherein generating the reference quantitative measure comprises normalizing to a median of medians for one or more molecules counts obtained from each sample in a plurality of samples.

11. The method of claim 1, wherein generating the reference quantitative measure comprises generating an expected log-number based on median of the log of normalized one or more molecule counts from a plurality of samples.

12. The method of claim 1, wherein comparing to the plurality of reference quantitative measures comprises normalizing to a median of medians for one or more molecules counts obtained from the test sample.

13. The method of claim 1, wherein comparing to the plurality of reference quantitative measures comprises subtraction of log 2 values for one or more molecules counts obtained from the plurality of samples from media-centered log 2 values for one or more molecule counts obtained from the test sample.

14. The method of claim 1, wherein the one or more loci are in a bin of 1-10 kb, 10-20 kb, 20-30 kb, 30-40 kb, 40-50 kb, 50-60 kb, 60-70 kb, 70-80 kb, 80-90 kb, 90-100 kb, 100-110 kb, 110-120 kb, 120-130 kb, 130-140 kb, 140-150 kb, 150 kb or more.

15. The method of claim 1, wherein determining an estimate of copy number variants (CNVs) for one or individual segments of the set of off-target sequence representations comprises comparison to a threshold.

16. The method of claim 1, wherein the threshold is based on maxima positive predictive accuracy (PPA) of the test sample and/or one or more additional samples.

17. The method of claim 1, wherein the threshold is based on maxima positive predictive accuracy (PPA) of the test sample and one or more additional samples.

18. The method of claim 1, wherein determining an estimate of copy number variants (CNVs) for one or individual segments of the set of off-target sequence representations comprises application of circular binary segmentation (CBS) to identify genomic segments of equal copy number.

19. The method of claim 1, further comprising determination of HRD status in a sample based on the estimate of CNVs for one or individual segments of the set of off-target sequence representations.

20. A system for performing the method of claim 1.

21. A computer readable medium comprising instructions for performing the method of claim 1.
